(19) United States
(12) Patent Application Publication
(10) Pub. No.: US 2002/0152482 A1
(43) Pub. Date: Oct. 17, 2002

(54) Mice with combined disruption of Gpx1 and Gpx2 genes have growth retardation, hypothermia, and colitis

(76) Inventors: Fong-Fong Chu, San Gabriel, CA (US); Robert S. Esworthy, San Gabriel, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 09/973,196

(22) Filed: Oct. 10, 2001

Related U.S. Application Data
(60) Provisional application No. 60/238,443, filed on Oct. 10, 2000.

(51) Int. Cl. A01K 67/027; C12N 5/06

(52) U.S. Cl. 800/9; 800/18; 435/354

(57) ABSTRACT

Disclosed is a transgenic knockout mouse whose genome has a homozygous disruption in its endogenous Gpx1 and Gpx2 genes, wherein the disruptions result in a decrease in GPX activity in the transgenic mice when compared to non transgenic mice of the same type. Methods for production of the mouse are presented. Also disclosed are cells derived from the transgenic knockout mouse. The mouse can be used in a method for identifying therapeutic agents for the treatment of an individual diagnosed with a metabolic disorder associated with a reduction or loss of expression of wild-type Gpx1 and Gpx2.

### CROSS REFERENCE TO RELATED APPLICATION

This application is related to provisional application No.60/238,443.

The publications and other materials used herein to illuminate the background of the invention or provide additional details respecting the practice are incorporated by reference.

### TECHNICAL FIELD OF THE INVENTION

The present invention is directed towards specific knockout(KO) animals and their use as animal models. More specifically, the knockout animals contain a disruption in the genes encoding GPX-1 and GPX-GI. Corresponding cells which are amenable to tissue culture are also part of the invention, as are methods of using such cells, including their use as a tool for identifying therapeutic agents.

### BACKGROUND OF THE INVENTION

Selenium-dependent glutathione peroxidases (GPXs) are a family of enzymes that are most efficient in the reduction of hydroperoxides. At the present time there are four known selenoproteins with GPX activity. These include the classical ubiquitous GPX-1, GPX-GI, the secreted GPX-P, and PHGPX (1). These GPX isozymes are encoded by four distinct genes, the Gpxl gene for GPX-1, the Gpx2 gene for GPX-GI, the Gpx3 gene for GPX-P, and the Gpx4 gene for PHGPX (2). The GPX-1 and GPX-GI isozymes have very similar properties including substrate specificity and cytosolic localization (3, 4). The unique feature of GPX-GI is its high levels of expression in the epithelium of the GI-tract. GPX-P is found in body fluids such as plasma, lung and GI-tract (5, 6). PHGPX is present at a high level in testis and is implicated in sperm maturation (1,7). PHGPX is present in low level in the GI-tract (8). Both GPX-P and PHGPX can reduce phospholipid and cholesterol hydroperoxides, while the latter is a more efficient enzyme at reducing these substrates (9, 10).

It is known that KO mice homozygous for disruption of single Gpx1 and Gpx2 genes (i.e., Gpx1-KO and Gpx2-KO mice) display little pathology without additional stress (11-13). However, aged Gpx1-KO mice display spontaneous weight loss (12). For example, the Gpx1 gene is expressed ubiquitously and is highly expressed in the erythrocyte, liver, and kidney. The antioxidant function of GPX-1 is revealed in Gpx1-KO mice especially after treatment with prooxidant chemicals. An increased level of H₂O₂ is produced from liver mitochondria in older (5-6 month) Gpx1-KO mice compared with that from wildtype mice without any treatment (12). GPX-1 can prevent lipid peroxidation induced by paraquat herbicide or measured in liver and lung and protect cortical neurons against H₂O₂ (14, 15). The Gpx1-KO mice are more susceptible to neurotoxic agents as malonate, 3-nitropropionic acid, and 1-methyl-4-phenyl-1,2,5,6-tetrahydropyridine (MPTP) in the brain (16). Malonate induces hydroxyl radical generation while MPP+, the active metabolite of MPTP, inhibits mitochondrial complex I activity (17). Since these neurotoxins produce oxidative stress and impair energy production, GPX-1 is implicated in protection against oxidative damage.

Depending on the type of insult, lack of GPX activity can also be beneficial under certain circumstances. Mice overexpressing GPX-1 and GPX-P have low levels of peroxides and prostaglandins and are more sensitive to hyperthermia (18). We have found that the jejunum crypt of Gpx1-KO mice regenerates better than that in the wildtype after exposure to high dose γ-irradiation (13). Perhaps the higher level of prostaglandins in the Gpx1-KO mouse intestine are directly responsible for crypt regeneration. Furthermore, the lack of GPX activity in the Gpx1-KO mouse can be protective against kainic acid-induced limbic seizures and neurodegeneration (19). This appears to result from decreased receptor function for N-methyl-D-aspartate (NMDA), since kainic acid induces NMDA-dependent seizure. Increased GPX activity in mice overexpressing the Gpx1 gene may have enhanced carcinogenic response in skin treated with 7,12-dimethylbenz[a]anthracene and 2—O-tetradecanoylphorbol-13-acetate (20). The mechanism of the pro-carcinogenic activity is not known but it is apparent that elevated antioxidant activity can be debilitating, depending on the type of insult.

GPX activity is also implicated in protection against infectious agents. For example, Jaeschke et al. have found that Gpx1-KO mice are more susceptible to neutrophil-mediated parenchymal cell injury during endotoxemia (21). The galactosamine/endotoxin induced acute liver failure involves neutrophils and GPX protects hepatocytes against peroxides generated by infiltrated neutrophils in the liver. It has also previously been shown that Gpx1-KO mice are more susceptible to coxsackievirus-induced myocarditis (22). Viral antibody titers in the Gpx1-KO mice are less than 20% of those found in the wildtype mice, suggesting that cellular immune response is impaired in the Gpx1-KO mice.

Gpx2-KO mice have also recently been generated and these mice appear to be normal (13). Unlike the Gpx1 gene, which is expressed ubiquitously, the Gpx2 gene is expressed specifically in epithelium. The Gpx2 gene is highly expressed in the gastrointestinal tract, and is also present in the breast, lung, and human liver (3, 23). In the GI-epithelium, GPX-1 and GPX-GI contribute to most of GPX activity (4). The lack of pathology in Gpx2-KO mice is not unexpected, since the Gpx2 gene has limited tissue expression, the Gpx1 gene is co-expressed in tissues expressing the Gpx2 gene, and GPX-1 and GPX-GI have similar biochemical and cellular properties.

Reactive oxidative species are implicated to play an important role in the pathogenesis of inflammatory bowel disease (IBD), which is caused, at least in part, by bacterial infection (24-26). IBD consists of two disorders that have similar symptoms, i.e., ulcerative colitis and Crohn's disease. Although elevated H₂O₂ is detected in IBD (27, 28), the protective effect of GPX against IBD has not yet been established. Elevated GPX activity in red blood cells and/or plasma found in IBD patients is implicated against the protective role of GPX in IBD (29, 30). Although selenium-deficiency is commonly present in those patients with severe gastrointestinal disorders (31, 32), this is believed to result from IBD rather than contributing to IBD.

The evaluation of chemical compounds for potential efficacy as human therapeutics necessitates data and information of a compounds efficacy in vivo. Ideally, the in vivo system would be human but ethical and pragmatic reasons prevent such data from being accumulated. As an alternative, many laboratory animals provide satisfactory systems for screening potential therapeutics for treating human physiological disorders. Recent advances in recombinant DNA technology have enabled researchers to genetically manipulate the genomes of animals to enhance such animal model systems. For example, the technique of transgenic generation have been utilized to produce knockout mice that do not express a particular endogenous gene. There presently exists a need for animal models which can be utilized to study the physiological function of GPX activity in the GI-tract. One approach to generate a useful model for such studies would be double knockout (double-KO) mice with a combined disruption of both alleles of each of the Gpx1 and Gpx2 genes.

### SUMMARY OF THE INVENTION

In accordance with the present invention, an animal model is provided for studying the significance of Gpx-1 and GPX-GI, in particular with regard to how these two gene products interact in animal physiology.

In one aspect, the invention provides transgenic mice deficient in both GPX-1 and GPX-GI activity. The deficiency is a result of a homozygous double knockout of the Gpx1 and Gpx2 genes in said transgenic mice.

In another aspect, the invention provides an animal model for the study of pathophysiological function of GPX activity in the ileum and colon in mammals. We have found that the homozygous double-KO mice of the present invention can exhibit symptoms associated with ileitis, colitis, growth retardation, hypothermia, wasting syndrome inflammatory bowel disease and cancer in the lower GI-tract.

In one aspect, the invention provides an animal model for the study of the degree of functional redundancy of GPX-1 and GPX-GI in the ileum and colon in mammals.

In one embodiment, the invention provides transgenic mice which have a homozygous knockout of the Gpx1 gene in said transgenic mice, together with a heterozygous knockout of one allele of the Gpx2 gene. The invention thus provides an animal model for the study of the degree of functional redundancy of GPX-1 and GPX-GI in the ileum and colon.

In another embodiment, the invention provides transgenic mice which have a homozygous knockout of the Gpx2 gene, together with a heterozygous knockout of one allele of the Gpx1 gene in said transgenic mice. The invention thus provides another animal model for the study of the degree of functional redundancy of GPX-1 and GPX-GI in the ileum and colon.

In another embodiment, the invention provides a transgenic double knockout mouse whose genome comprises a homozygous disruption of the endogenous Gpx1 gene and a homozygous disruption of the endogenous Gpx2 gene, wherein each disruption comprises the insertion of a transgene, and wherein the combined disruptions result in a decreased level of GPX-1 and GPX-GI production and decreased number of cells producing GPX-I and GPX-GI in the transgenic mouse as compared to a nontransgenic mouse.

In another embodiment, the invention provides a transgenic double knockout Gpx1/Gpx2 mouse which exhibits a physiological disease, symptom or symptoms selected from the group consisting of ileitis, colitis, hypothermia, decreased rate of weight gain, perianal ulceration, diarrhea, wasting syndrome, inflammatory bowel disease and cancer of the lower gastro-intestinal tract.

In another embodiment, the invention provides a cell or cells isolated from a double knockout Gpx1/Gpx2 mouse.

In one embodiment, the invention provides a transgenic double knockout Gpx1/Gpx2 mouse which further comprises a mouse which is germ free.

In another embodiment, the invention provides the double knockout of the Gpx1 and Gpx2 genes in mice having different genetic backgrounds. The invention thus provides means to identify other genes that affect the severity of IBD symptoms and progression to cancer.

In another embodiment, the invention provides the double knockout of the Gpx1 and Gpx2 genes in a genetic background of a B6 mouse.

In another aspect, the invention provides a method of selecting an agent for treating a metabolic disorder selected from the group consisting of: ileitis, colitis, hypothermia, decreased rate of weight gain, perianal ulceration, diarrhea, wasting syndrome, inflammatory bowel disease and cancer of the lower gastro-intestinal tract comprising:

(a) measuring a symptom in a knockout mouse whose genome is manipulated to comprise a homozygous disruption of both the endogenous Gpx1 and Gpx2 genes, wherein the disruption of both the Gpx1 and Gpx2 genes results in said knockout mouse exhibiting one of said disease, symptom or symptoms;

(b) administering an agent to said mouse;

(c) measuring one or more of said symptoms in the mouse after administering the agent; and

(d) comparing at least one of said symptoms in the mouse before and after administering the agent, wherein a decrease in said disease, symptom or symptoms after administering the agent indicates the agent is an agent for treating said disease, symptom or symptoms associated with a metabolic disorder.

In another embodiment, the method of observing the effects of treatment of a disease, symptom or symptoms in double-KO Gpx1 and Gpx2 transgenic mice by administering an agent is observed and compared in double-KO Gpx1 and Gpx2 mice having different genetic backgrounds. The method comprises:

(a) measuring a symptom in a first double knockout mouse having a first genetic background, whose genome is manipulated to comprise a homozygous disruption of both the endogenous Gpx1 and Gpx2 genes, wherein the disruption of both the Gpx1 and Gpx2 genes results in said knockout mouse exhibiting a disease, symptom or symptoms selected from the group consisting of: ileitis, colitis, hypothermia, decreased rate of weight gain, perianal ulceration, diarrhea, wasting syndrome, inflammatory bowel disease and cancer of the lower gastro-intestinal tract;

(b) measuring said symptom in a second double knockout mouse having a second genetic background, whose genome is manipulated to comprise a homozygous disruption of both the endogenous Gpx1 and Gpx2 genes, wherein the disruption of both the Gpx1 and Gpx2 genes results in said knockout mouse exhibiting at least one of said disease, symptom or symptoms;

(c) administering an agent to said first and second mouse;

(d) measuring one or more of said symptoms in the first and second mouse after administering the agent; and

(e) comparing at least one of said symptoms in said first and second mouse before and after administering the agent, wherein a decrease in said disease, symptom or symptoms after administering the agent indicates the agent is an agent for treating said disease, symptom or symptoms associated with a metabolic disorder.

In another embodiment, the invention provides a method of observing the effects of treatment of a disease, symptom or symptoms in single and double-KO GPX transgenic mice in mice with a B6 genetic background.

In another embodiment, the invention provides a method of selecting an agent that modulates GPX enzyme activity comprising:

(a) administering an agent to a first group of isolated mouse intestinal epithelial cells and not to a second group of mouse intestinal epithelial cells, wherein the genome of both the first and second isolated mouse cell groups has been manipulated to comprise a homozygous disruption of both alleles of the endogenous Gpx1 gene and Gpx2 genes, and wherein the homozygous disruption of both the endogenous Gpx1 gene and Gpx2 genes prevents expression of functional GPX proteins; and

(b) determining the amount of GPX enzyme activity of the first and second cell groups, wherein a difference in the amount of proliferation of the first cell group as compared to the second cell group indicates that the agent modulates GPX enzyme activity.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1A depicts the results of Southern analysis of various Gpx1 and Gpx2 genes.

FIG. 1B depicts the results of GPX activity in Gpx1 and Gpx2 double knockout mice compared to non double-KO littermates.

FIG. 2A is a graphical representation of the growth (in body weight) of double-KO mice compared with their non-double-KO littermates.

FIG. 2B is a graphical representation of the age at which double-KO mice show growth retardation.

FIG. 3A is a graphical representation of body temperature of double-KO mice compared to non-double-KO littermates.

FIG. 3B is a graphical representation of body temperature in response to stress of double-KO mice compared to non-double-KO littermates.

FIG. 4 is a photograph of histological preparations of double-KO mice compared to non-double-KO littermates.

### DETAILED DESCRIPTION OF THE INVENTION

General Methods

The practice of the present invention employs, unless otherwise indicated, conventional techniques of chemistry, molecular biology, microbiology, recombinant DNA, genetics, immunology, cell biology, cell culture and transgenic biology, which are known to one of ordinary skill in the related art.

Gpx1-KO mice were generated by using standard techniques as C57BL6/J (96) and 129Sv/J hybrids and B6 inbred mice as described previously (11). The generation of Gpx2-KO mice as B6 and 129S3 hybrids and B6 mice has been described previously (13). These mice were housed in ventilated cage racks (Allentown Caging Equipment, Allentown, N.J.) under conventional housing conditions. The colony was monitored for infectious pathogens via sentinel mouse surveillance and necropsy of randomly selected littermates of the double-KO mice. The loose stools of several double-KO mice were negative for parasites. All mice had free access to laboratory rodent diet (5001, Purina Mills Inc., Richmond, Ind.) and water. This diet contains 23% protein, 4.5% fat, 6% fiber, and 0.28 ppm selenium as provided by the manufacturer (http://www.labdiet.com).

Genotyping of Gpx1 and Gpx2-KO mice was done with either Southern or PCR analysis of DNA isolated from tails. For Southern analysis, 10 μg DNA was digested with BamHI or ApaI to determine the genotype of Gpx1 and Gpx2, respectively. After overnight digestion, DNA was resolved in 0.75% agarose gel and transferred to Zeta Probe membrane (BioRad Lab., Richmond, Calif.) and probed with ³²P-labeled and random-primed 3′ EcoRI fragment of mouse Gpx1 cDNA and mouse Gpx2 exon 2 CDNA. The Southern blot was analyzed by phosphor imaging (Molecular Dynamics, Sunnyvale, Calif.) (13). Polymerase Chain Reactions (PCR) were also performed. The PCR primers for the wildtype Gpx1 allele were mPX101F (DNA SEQ ID NO. 1:5′-AAGGAGGTGCAGGCGGCTGTGAGCG-3′) and GPX15 (SEQ ID NO. 2:5′-ACCGTTCACCTTGCACTTCTC-3′), which amplified about 600 bp DNA fragment. The primers for the Gpx1-KO allele were pPNTpgk (SEQ ID NO. 3:5′-CAGTTTCATAGCCTGAAGAACGAGAT-3′) and GPX15, which amplified a^{˜} 200 bp DNA fragment. The primers for the wildtype Gpx2 allele were MPX206 (SEQ ID NO. 4:5′-CCCACCTGTCTAGAGGACTTA-3′) and MPXin09 (SEQ ID NO. 5:5′-TCCATGCCAACGTAGTGATT-3′), which amplified a ^{˜}600 bp DNA. The primers for the Gpx2-KO allele were MPX206 and pPNTpgk, which amplified a ^{˜}400 bp DNA. Both alleles were amplified in the same reaction tubes.

Metabolic Studies

Rectal temperature was measured with Thermalert mouse probe (Model TH-8, Physitemp Instrument Inc., Clifton, N.J.) at the 6-8 am on mice under normal housing. To quantify the food and water consumption and feces and urine output mice were placed in metabolic cages without bedding for 24 h. This setting appeared to be stressful for the double-KO mice, as shown by frequent hunched-over appearance, pilocrection of their coat, and loose stools the next day.

Histology of Small and Large Intestine

Mice were sacrificed by halothane overdose (Halocarbon Labs, North Augusta, S.C.). After removing the lumen contents, sections of jejunum, ileum, colon, and rectum were rinsed with phosphate buffered saline, and then fixed in 10% buffered formalin or Bouin's fixative for 2-3 h. The tissues were then dehydrated in ethanol, and embedded in paraffin and sectioned onto slides. The tissue sections were stained with hematoxylin and eosin (H&E) alone or in addition to periodic acid Schiff (PAS) staining.

GPX Activity Assay

GPX activity was determined on mouse intestinal and colon epithelium. Jejunal and ileal epithelium were isolated from the proximal and the distal one third of small intestine as described previously (13). The GPX activity was measured with 60 μM H₂O₂ and 3 mM GSH at pH 7.3. The protein concentration was determined with a BCA assay (Pierce Chemical, Rockford, Ill.) with bovine serum albumin as the standard.

### EXAMPLES

The invention is further illustrated by the following examples, which are not intended to be limiting.

### Example 1

Generation of Double-KO Mice

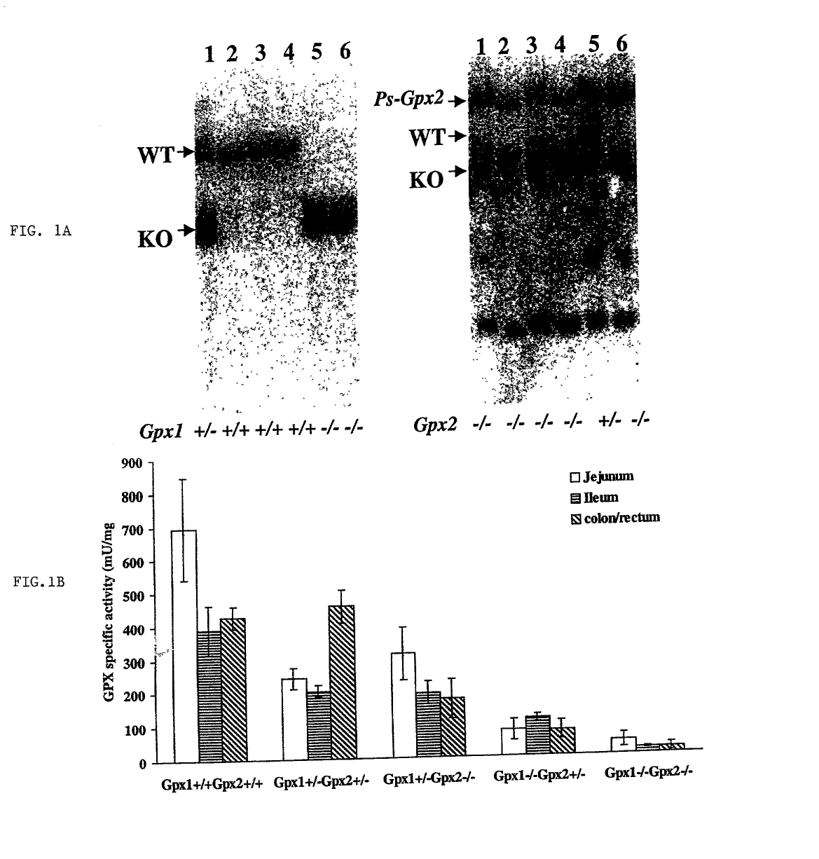

Homozygous Gpx1-KO and Gpx2-KO mice were bred to generate heterozygous double-KO mice. These heterozygous double-KO mice were bred to each other, one sixteenth of the offspring were homozygous double-KO mice. One half of mice were reciprocal homozygous and heterozygous KO's, so called 3-quarter KO's. These double-KO and 3-quarter KO were then used as breeders to generate double-KO mice. The genotypes of six mice were analyzed by Southern analysis to examine genetic characteristics of the results of double knockout breeding. Referring now to FIG. 1A, the left panel contains BamHI-digested DNA hybridized with mouse Gpx1 cDNA. The top arrow points at ^{˜}11 kb wildtype (WT) allele, and the lower arrow points at ^{˜}4.3 kb Gpx1-KO allele. The right panel contains ApaI-digested DNA hybridized with mouse Gpx2 cDNA. The top arrow points at ^{˜}14 kb Gpx2 pseudogene (Ps-Gpx2), the middle arrow points at ^{˜}7 kb WT allele, and the lowest arrow points at 4.9 kb Gpx2-KO allele. The other two DNA fragments of low molecular weights do not correlate with Gpx2 genotypes, and are ignored. The genotypes are shown in the bottom of the panels and are designated as follows:+/−, one wildtype and one knockout allele; +/+two wildtype alleles; −/−, two knockout alleles.

Referring now to FIG. 1B, there are shown the results of GPX enzyme activity in the epithelium of mouse lower GI-tract. GPX activity was measured using hydrogen peroxide as the substrate. The error bars represent variances or standard deviations of the means. The number of mice assayed in each group from left to right is 3, 2, 4, 4, and 4 respectively. The Genotypes for both Gpx1 and Gpx2 are as in 1A.

The number of the double-KO mice was close to the predicted value from Mendelian genetics. Similar numbers of male and female offspring were obtained. This indicates that the double-KO mice have normal embryonic development and there is no gender bias. Both male and female double-KO mice can be fertile but only a small percentage of mice gain enough weight and appear healthy enough to be used as breeders.

### Example 2

Growth of Double-KO Mice

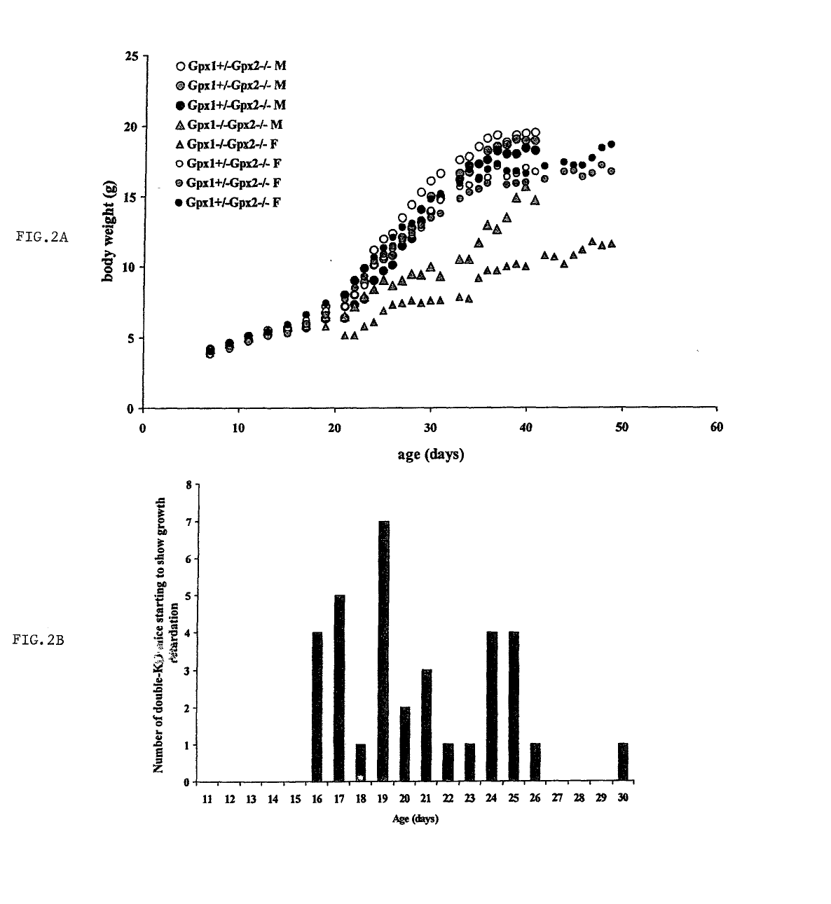

Referring now to FIG. 2A, there are shown the results of growth activity in adult (45-47 days old) homozygous double-KO mice. There is shown a graphical representation of the growth rate of a single litter of 8 pups. Male mice are shown in larger symbols, and female mice are shown in smaller symbols. Circles represent Gpx1+/−Gpx2−/−mice, diamonds represent Gpx1−/−Gpx2+/−mice, squares represent Gpx1+/−Gpx2+/−mice, and triangles represent Gpx1−/−Gpx2−/−mice. The female and male double-KO mice in the top panel started to show growth retardation at 21 and 26 days old.

Referring now to FIG. 2B, there is shown a graphical representation of the number and age of 33 homozygous double-KO mice at which they first show growth retardation. The double-KO had almost background GPX activity in the mucosa of small and large intestine (Lower Panel of FIG. 1). Since the jejunum mucosa had a high level of GPX-1 and low level of GPX-GI as shown previously (4), the total GPX activity in this region corresponded only to the Gpx1 gene dosage. The GPX-GI contributed little to GPX activity in the jejunum even in a homozygous Gpx1-KO background since the heterozygous and homozygous Gpx2-KO mice do not have statistically different GPX activity (P=0.10) as shown in the last two groups in FIG. 1A. A lower level of GPX-1 and a higher level of GPX-GI are expressed in the ileal mucosa compared with that in jejunal mucosa. The dosage effect of the Gpx2 allele is evident only in the absence of Gpx1 gene expression. In colon mucosa, the heterozygous double-KO has the same level of GPX activity as wildtype mice.

Gross Phenotypes of Double-KO Mice

The homozygous double-KO mice had a slower weight gain compared with mice of other genotypes starting around day 16 postnatally. The two double-KO mice had the same birth weight and maintained the same weight gain as their littermates until weaning. Among the 33 double-KO mice followed, 32 showed growth retardation onset at 16-26 days old. The last one started to show growth retardation at 30 days.

Other symptoms often associated with these homozygous double-KO mice include perianal ulceration (redness and irritation of anal region), anal mucous discharge, and diarrhea. One or more of these symptoms occurred as early as 14 days old. However, most of these symptoms were transient except the perianal ulceration, which appeared to be persistent. Older double-KO transgenic mice, over six months old, had a high level of tumor in the ileum.

The younger homozygous double-KO mice had at least 25% mortality. Death or morbidity indicating imminent death occurred between 20-36 days of age. Five of the 33 homozygous mice that we tracked daily died unexpectedly, three more of the 33 mice were terminated when they appeared moribund judging by persistent weight loss, hunched-over posture, or rectal obstruction. No noticeable abnormality was seen in major organs, for example such the liver, kidney, heart, lung, spleen or lymph nodes in the autopsy.

In spite of the severe growth retardation, wasting syndrome, and mortality, the homozygous double-KO mice had similar weight and length of small and large intestine compared with their littermates up to 25 days old. After 40 days, the length and weight of small intestine in the homozygous double-KO mice began to lag behind their littermates by 20%. However, the weight of colon and rectum in the homozygous double-KO mice was about 20% heavier than that in their littermates. This may simply reflect the thickening of colon mucosa in the double-KO mice.

### Example 3

Rectal Hypothermia in Double-KO Mice

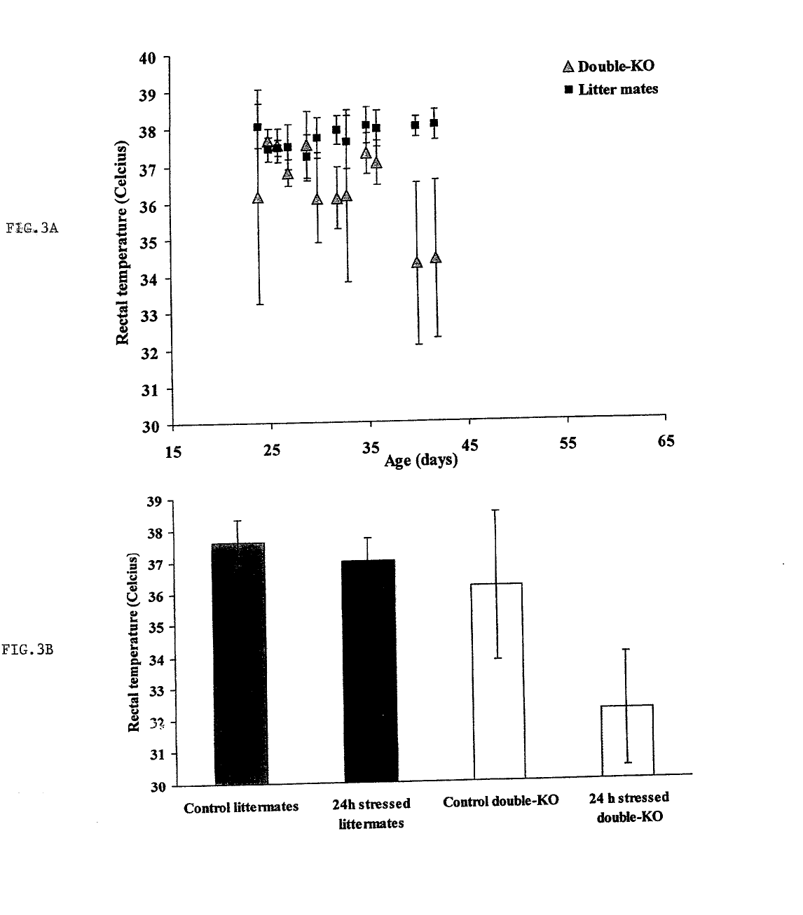

To determine if the severe growth retardation was contributed by lack of calorie uptake despite of normal intestinal growth in the homozygous double-KO mice, we monitored the rectal temperature and amount of food uptake by these mice. We found these mice are hypothermic compared with their littermates either under normal housing condition or in metabolic cages where there was no bedding. Referring now to FIG. 3A, there is shown a graphical representation of rectal temperatures of double-KO mice as compared to their littermates. Rectal temperature of homozygous double-KO mice and their littermates with either combined heterozygous KO or three-quarter KO. Double-KO mice are triangles and their littermates are squares. The error bars are variances or standard deviations from means of 2-6 mice. Rectal temperatures of the younger (24-36 days old) and more mature (40-67 days old) double-KO mice were 37.0±1.1° C. and 35.1±2.2° C. respectively. The rectal temperatures of their littermates were 37.6±0.6° C. for all ages under normal housing condition. After being placed in metabolic cages for 24 h, the rectal temperature of 36 day-old double-KO mice had dropped from 36.2±2.3° C. to 32.2±1.8° C. as shown in FIG. 3B. The control mice did not change their rectal temperature significantly after being housed in metabolic cages. The homozygous double-KO mice (24-49 days old) consumed similar amounts of food (0.16±0.07 g mouse chow/g body weight per day, n=11) as their littermates (0.10±0.05 g chow/g body weight per day, n=18). The difference in food intake is not statistically significant. Although the animals had bouts of acute diarrhea and loose stools, they did not have chronic diarrhea.

Referring now to FIG. 3B, there is shown a graphical representation of hypothermia caused by stress in double-KO mice. Adult (36-day-old) mice were stressed by housing singly or doubly in metabolic cages for 24 hours. The error bars are variances of means from four double-KO mice and six littermates with Gpx1+/−Gpx2+/− and Gpx1−/−Gpx2+/−genotypes.

### Example 4

Inflammation of the Small Intestine and Colon/Rectum

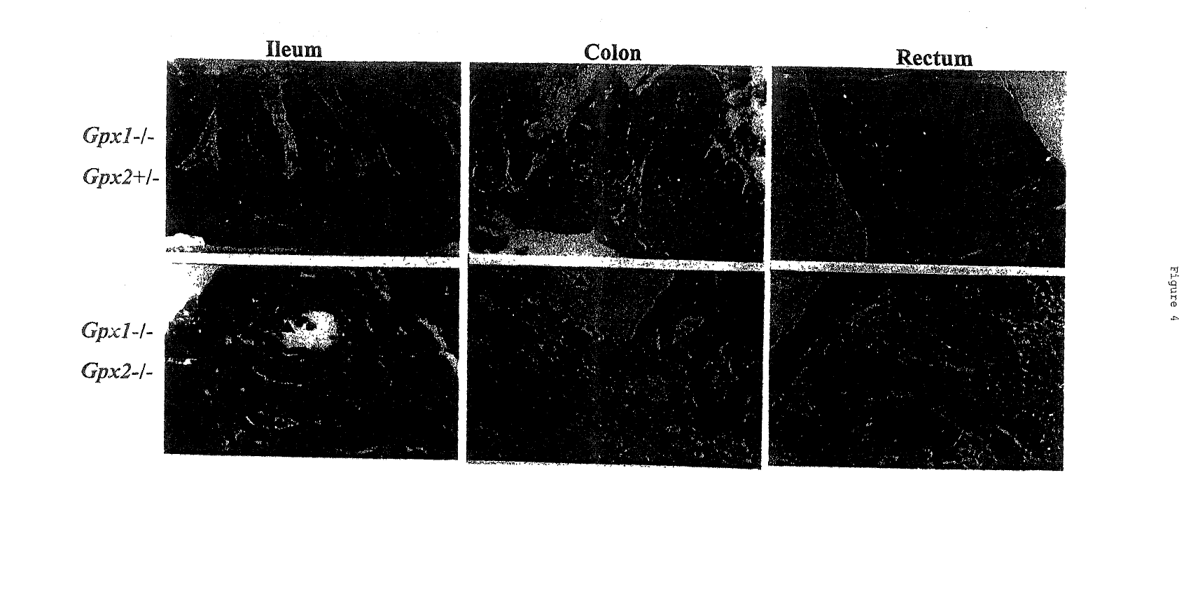

Histological analysis was performed on the cross sections of stomach, jejunum, ileum, colon and rectum after staining with hematoxylin and eosin as shown in FIG. 4. Cross sections from two 20 day-old littermates with homozygous double-KO and 3-quarter KO genotypes were compared. The 3-quarter KO had apparent normal histology throughout the GI-tract. In contrast, the double-KO mouse had severe ileitis and colitis, although the jejunum and stomach appeared to be unaffected. Crypt abscesses were prevalent in ileum, colon and rectum.

The extent of ileitis and colitis were scored with the histological changes in five categories: (1) severity of the inflammatory cell infiltrate in lamina propia; (2) epithelial cell reactive hyperplasia/atypia; (3) mucin depletion (colon and rectum only); (4) increases in intraepithelial lymphocyte numbers in crypts; and (5) number of inflammatory foci as defined previously (33). Periodic acid Shift (PAS) staining was performed on some sections to confirm the depletion of mucin. Referring now to FIG. 4, there are shown the results of histology of mouse ileum, colon and rectum stained with eosin and hematoxylin. One 3-quarter KO (top row) and one homozygous double-KO (lower row) littermates were sacrificed at 20 days of age. Arrows point at crypt abscesses. The original magnification is 200×.

Table 1 shows the progression of ileitis and colitis from distal to proximal direction in 18 homozygous double-KO mice through early development.

TABLE 1
Severity of inflammation in double-KO mice of the
present invention.

| | Age (days) | | | Total no. of |
|---|---|---|---|---|
| | 11-14 | 15-17 | 20-27 | mouse |
| Jejunum^{a} | 7N^{b} | 3N | 5N | 15 |
| Ileum^{a} | 7N | 3N | 4M^{b}, 4S^{b} | 18 |
| Proximal colon^{a} | 6N, 1S | 3S | 1N, 2M, 5 | S18 |
| Distal colon^{a} | 1N, 3M, 3S | 3S | 2M, 6S | 18 |

^{a}jejunum was sampled at 2-4 cm from stomach; ileum was sampled within 2 cm of cecum; promixal colon was taken within 2 cm of cecum; and distal colon was taken within 1-2 cm from anus.
^{b}N = normal (with 0-1 scores, the same as control mice); M = mild colitis (with 4.5-7 scores); S = severe (with 8-10 scores). Scoring was based on the 14 point system described by Aranda et al. (33): inflammation (0-3 points), mucin depletion (0-2 points), reactive epithelium (0-3), number of intraepithelial lymphocytes (0-3), inflammatory foci (0-3). No samples were scored in the range of 2-4 or greater than 10.

Spontaneous colitis was shown mostly in the distal colon as early as 11 days of age in 6 out of seven mice analyzed. The proximal colitis was only observed in one of seven 11-14 days old mice analyzed. Most mice of 15 days and older had inflammation in both distal and proximal colon. Ileitis became evident and prevalent in mice of 20-27 days old. No inflammation was seen in the stomach and jejunum in all animals up to 60 days old (Table 1 and other observations). Other major organs including heart, liver, lung, kidney, testis, and brain did not have any noticeable abnormality upon gross and histological analysis.

Mice with disrupted single Gpx1 and Gpx2 genes are apparently normal. This raises some question as to the individual importance of each of these antioxidant enzymes. This lack of an observable deleterious phenotype in single knockout mice also suggests that animals have overlapping defense system against hydroperoxides, since catalase, glutathione S-transferases and AOP-2 can reduce some species of GPX substrates (34-37). In contrast to the single knockout mice, the gross abnormality found in mice with combined disruption of Gpxl and Gpx2 genes demonstrates the uniqueness of GPX activity' which cannot be compensated by other types of hydroperoxide-reducing enzymes. This result also suggests that GPX-1 and GPX-GI are functionally redundant.

The GPX-1 appears compensating for lack of GPX-GI in epithelium of small intestine judged by the same level of GPX activity detected in mice expressing 0 and 1 Gpx2 allele. A higher level of GPX-1 in homozygous Gpx2-KO intestine was detected compared to the GPX-1 level in wildtype mice determined by immunoprecipitation (4, 13). The same level of GPX-GI was detected in Gpx1-KO intestinal mucosa. These observations suggest the Gpx1 gene compensates for lack of Gpx2 gene expression, but not vice versa. The compensation appears to be limited to small intestine but not colon. Alternatively, it is also possible that a part of the expression machinery necessary for selenoproteins in favor of Gpx1 but not Gpx2 gene expression is active in the intestine but not in colon epithelium. This selenoprotein expression machinery includes 3′-untranslated region selenocysteine insertion sequence (SECIS) in mRNA (38), selenocysteine tRNA[Ser]sec (39), a SECIS binding protein named SBP2 (40, 41), and mammalian Upfl protein (also known as Rent or regulator of nonsense transcripts) (42), etc. It is not clear if any of these factors differentiate between Gpx1 and Gpx2 mRNAs. The same GPX level in colon mucosa of wildtype control and heterozygous double-KO mice suggests that this expression machinery for selenoproteins may be a limiting factor.

It is clear that the double-KO mice have almost no GPX activity in the mucosa of distal GI-tract. Although 3-quarter KO mice with no Gpx1 alleles have only a small fraction of total GPX activity in the distal GI-tract, this low level of activity appears to sufficient to maintain normal physiology. In fact, rodent GI-epithelium may have one-fold higher GPX activity compared with that in humans. The specific activity of GPX in human intestine and colon mucosa is 100-240 mU/mg protein compared with 300-700 mU/mg in rats (4) and mice. Although the difference in GPX activity level in the GI-tract is not as big as that in liver, where humans have 352+89 mU/mg (43) and rodents have ˜4,000 mU/mg (44, 45), the lower GPX activity level in human GI-tract suggests its higher susceptibility to peroxidative injury.

The first sign of abnormality observed in these double-KO mice is growth retardation. It is well documented that severe Se-deficiency causes growth retardation in young animals (46). Injection of triiodothyronine (T₃) to restore plasma thyroid levels in these Se-deficient animals did not increase animal weight gain (47). Since GPXs are Se-dependent enzymes, this slow growth caused by Se-deficiency in 2^{nd} generation rodents can be explained by lack of GPX-1 and GPX-GI in the GI-tract. This suggests that these 2^{nd} generation Se-deficient animals should be examined for colitis. To determine if growth retardation in the homozygous double-KO mice is due to lack of food intake, mice were placed in metabolic cages to monitor the amount of food, water and excretion for a 24-hour period. Often, two mice were placed in one metabolic cage since the double-KO mice could not sustain the stress well when housed alone in this setting. The stress may be contributed by the cooler air due to lack of bedding and shelter. Since the double-KO mice consume the same amount of food as their littermates, and do not have chronic osmotic diarrhea, it is possible that these double-KO mice are either deficient in converting the calorie intake into metabolic fuel as implicated in the older Gpx1-KO mice (12), or suffering from inflammation-induced cachexia (48).

Many mammals respond to energy deficit, such as calorie restriction, by lowering body temperature (49). In fact, fasting can induce torpor or extreme hypothermia in mice (50). Since these mice have wasting syndrome, we wanted to determine if they also have hypothermia consistent with deprivation in metabolic energy. The hypothermia presented in these mice support the notion that these mice may not be getting enough calories despite unrestricted access to food and normal appetite. It will readily be appreciated by those skilled in the art that determination of hypothermia in mice of the present invention supplied with a high fat diet can be utilized to answer this question.

Animals suffering from inflammatory bowel disease (IBD) often show wasting (33). The double-KO mice tend to have colon inflammation, which shows up as a thickened colon and heavier colon weight (24). Histological study shows that these homozygous double-KO mice have spontaneous inflammation starting from distal colon as early as 11 days old, which was the youngest age analyzed. Inflammation progresses from distal colon to proximal colon and then to ileum. The increased severity in colonic inflammation around weaning appears to be correlated with the increased number of species of colonic bacteria (25). The alteration in colonic bacterial flora can result from either ingestion of solid food which alters luminal pH (51) or decreased in the protective IgA and other bactericidal components present in milk (52). The severity and timing of ileitis and colitis in these double-KO mice is consistent with the notion that microflora is an important cofactor in the pathogenesis of colonic inflammation (24, 25). Germ free mice of the present invention can be utilized to make this analysis.

Reactive oxygen species have been implicated in the pathogenesis of IBD. The inflamed colon has elevated levels of oxygen metabolites detected by chemiluminescence (27, 28). Catalase, superoxide dismutase, or azide (a myoleperoxidase inhibitor) decreases chemiluminescence. Compounds used for IBD therapy such as 5-aniinosalicylates have antioxidant activity (26, 53). Thus, increased oxidative stress may play an important role in the pathogenesis of IBD. Selenium deficiency is common in those patients with severe gastrointestinal disorders due to impaired intestinal absorption (31, 32). However, little evidence supports the role of GPX in pathogenesis of IBD. Thus, the IBD phenotypes presented in these double-KO mice provide the first evidence to link GPX activity with this disease affecting one million Americans.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### References

1. Ursini, F., Heim, S., Kiess, M., Maiorino, M., Roveri, A., Wissing, J. & Flohe, L. (1999) Science 285, 1393-6.

2. Chu, F. F. (1994) Cytogenet Cell Genet 66, 96-8.

3. Chu, F. F., Doroshow, J. H. & Esworthy, R. S. (1993) J Biol Chem 268, 2571-6.

4. Esworthy, R. S., Swiderek, K. M., Ho, Y. S. & Chu, F. F. (1998) Biochim Biophys Acta 1381,213-26.

5. Tham, D. M., Whitin, J. C., Kim, K. K., Zhu, S. X. & Cohen, H. J. (1998) Am J Physiol 275, G1463-71.

6. Kim, K. K., Whitin, J. C., Sukhova, N. M. & Cohen, H. J. (1999) Pediatr Res 46, 715-21.

7. Roveri, A., Casasco, A., Maiorino, M., Dalan, P., Cafligaro, A. & Ursini, F. (1992) J Biol Chem 267, 6142-6.

8. Chu, F. F. & Esworthy, R. S. (1995) Arch Biochem Biophys 323, 288-94.

9. Thomas, J. P., Maiorino, M., Ursini, F. & Girotti, A. W. (1990) J Biol Chem 265, 454-61.

10. Esworthy, R. S., Chu, F. F., Geiger, P., Girotti, A. W. & Doroshow, J. H. (1993) Arch Biochem Biophys 307, 29-34.

11. Ho, Y. S., Magnenat, J. L., Bronson, R. T., Cao, J., Gargano, M., Sugawara, M. & Funk, C. D. (1997) J Biol Chem 272, 16644-51.

12. Esposito, L. A., Kokoszka, J. E., Waymire, K. G., Cottrell, B., MacGregor, G. R. & Wallace, D. C. (2000) Free Radic Biol Med 28, 754-66.

13. Esworthy, R. S., Mann, J. R., Sam, M. & Chu, F. (2000) Am J Physiol Gastrointest Liver Physiol 279, G426-G436.

14. Cheng, W. H., Ho, Y. S., Valentine, B. A., Ross, D. A., Combs, G. F., Jr. & Lei, X. G. (1998) J. Nutr 128,1070-6.

15. de Haan, J. B., Bladier, C., Griffiths, P., Kelner, M., O'Shea, R. D., Cheung, N. S., Bronson, R. T., Silvestro, M. J., Wild, S., Zheng, S. S., Beart, P. M., Hertzog, P. J. & Kola, 1. (1998) J Biol Chem 273, 22528-36.

16. Klivenyi, P., Andreassen, 0. A., Ferrante, R. J., Dedeoglu, A., Mueller, G., Lancelot, E., Bogdanov, M., Andersen, J. K., Jiang, D. & Beal, M. F. (2000) J Neurosci 20, 1-7.

17. Tipton, K. F. & Singer, T. P. (1993) J Neurochem 61, 1191-206.

18. Mirochnitchenko, O., Palnitkar, U., Philbert, M. & Inouye, M. (1995) Proc Natl Acad Sci U S A 92, 8120-4.

19. Jiang, D., Akopian, G., Ho, Y. S., Walsh, J. P. & Andersen, J. K. (2000) Exp Neurol 164, 257-68.

20. Lu, Y. P., Lou, Y. R., Yen, P., Newmark, H. L., Nfirochnitchenko, 0. I., Inouye, M. & Huang, M. T. (1997) Cancer Res 57, 1468-74.

21. Jaeschke, H., Ho, Y. S., Fisher, M. A., Lawson, J. A. & Farhood, A. (1999) Hepatology 29,443-50.

22. Beck, M. A., Esworthy, R. S., Ho, Y. S. & Chu, F. F. (1998) Faseb J 12,1143-9.

23. Chu, F. F., Esworthy, R. S., Lee, L. & Wilczynski, S. (1999) J Nutr 129, 1846-1854. 24. Panwala, C. M., Jones, J. C. & Viney, J. L. (1998) J Immunol 161, 5733-44.

25. Madsen, K. L., Doyle, J. S., Tavemini, M. M., Jewell, L. D., Rennie, R. P. & Fedorak, R. N. (2000) Gastroenterology 118, 1094-105.

26. Sands, B. E. (2000) Gastroenterology 118, S68-82.

27. Simmonds, N. J., Allen, R. E., Stevens, T. R., Van Someren, R. N., Blake, D. R. & Rampton, U.S. (1992) Gastroenterology 103, 186-96.

28. Keshavarzian, A., Sedghi, S., Kanofsky, J., List, T., Robinson, C., Ibrahim, C. & Winship, D. (1992) Gastroenterology 103,177-85.

29. Thomas, A. G., Miller, V., Shenkin, A., Fell, G. S. & Taylor, F. (1994) J Pediatr Gastroenterot Nutr 19, 213-9.

30. Hoffenberg, E. I., Deutsch, J., Smith, S. & Sokol, R. J. (1997) Am J Clin Nutr 65, 1482-8. 31. Rannem, T., Ladefoged, K., Hylander, E., Hegnhoj, J. & Jarnum, S. (1992) Am J Clin Nutr 56, 933-7.

32. Rannem, T., Ladefoged, K., Hylander, E., Hegnhoj, J. & Staun, M. (1998) Scand J Gastroenterol 33, 1057-61. 33. Aranda, R., Sydora, B. C., McAllister, P. L., Binder, S. W., Yang, H. Y., Targan, S. R. & Kronenberg, M. (1997) J Immunol 158, 3464-73.

34. Jakoby, W. B. (1985) Methods Enzymol 113,495-9.

35. Kang, S. W., Chae, H. Z., Seo, M. S., Kim, K., Baines, I. C. & Rhee, S. G. (1998) J Biol Chem 273, 6297-302.

36. Fisher, A. B., Dodia, C., Manevich, Y., Chen, J. W. & Feinstein, S. 1. (1999) J Biol Chem 274, 21326-34. 37. Esworthy, R., Chu, F. F., and Doroshow, J. H. (1999) in Current Protocols in Toxicology, ed. Maines, M., Costa, L., Reed, D., and Sassa, S. (John Wiley & Sons, Inc., pp. 7.1.1-7.1.32.

38. Gasdaska, J. R., Hamey, J. W., Gasdaska, P. Y., Powis, G. & Berry, M. J. (1999) J Biol Chem 274, 25379-25385.

39. Moustafa, M. E., El-Saadani, M. A., Kandeel, K. M., Mansur, D. B., Lee, B. J., Hatfield, D. L. & Diamond, A. M. (1998) Rna 4,1436-43.

40. Copeland, P. R. & Driscoll, D. M. (1999) J Biol Chem 274, 25447-54.

41. Copeland, P. R., Fletcher, J. E., Carlson, B. A., Hatfield, D. L. & Driscoll, D. M. (2000) Embo J 19, 306-314.

42. Sun, X., Perlick, H. A., Dietz, H. C. & Maquat, L. E. (1998) Proc Natl Acad Sci U S A 95,10009-14.

43. Esworthy, R. S., Baker, M. A. & Chu, F. F. (1995) Cancer Res 55, 957-62.

44. Chu, F. F., Esworthy, R. S., Ho, Y. S., Bermeister, M., Swiderek, K. & Elliott, R. W. (1997) Biomed Environ Sci 10, 156-62.

45. Esworthy, R. S., Ho, Y. S. & Chu, F. F. (1997) Arch Biochem Biophys 340, 59-63.

46. Thompson, K. M., Haibach, H., Evenson, J. K. & Sunde, R. A. (1998) J Nutr 128, 1289-95.

47. Thompson, K. M., Haibach, H. & Sunde, R. A. (1995) J Nutr 125, 864-73.

48. Liu, Z., Geboes, K., Colpaert, S., Overbergh, L., Mathieu, C., Heremans, H., de Boer, M., Boon, L., DHaens, G., Rutgeerts, P. & Ceuppens, J. L. (2000) J Immunol 164, 6005-14.

49. Lane, M. A., Baer, D. J., Rumpler, W. V., Weindruch, R., Ingram, D. K., Tilmont, E. M., Cutler, R. G. & Roth, G. S. (1996) Proc Natl Acad Sci U S A 93, 4159-64.

50. Gavrilova, O., Leon, L. R., Marcus-Samuels, B., Mason, M. M., Castle, A. L., Refetoff, S., Vinson, C. & Reitman, M. L. (1999) Proc NatlAcadSci USA 96,14623-8.

51. Hentges, D. J., Marsh, W. W., Petschow, B. W., Thai, W. R. & Carter, M. K. (1992) J Pediatr Gastroenterol Nutr 14, 146-52.

52. Orlando, S. (1995) J Obstet Gynecol Neonatal Nurs 24, 678-83.

53. Nfillar, A. D., Rampton, D. S., Chander, C. L., Claxson, A. W., Blades, S., Coumbe, A., Panetta, J., Morfis, C. J. & Blake, D. R. (1996) Gut 39,407-15.

What is claimed is:

1. A transgenic double knockout mouse whose genome comprises a homozygous disruption of the endogenous Gpx1 gene and a homozygous disruption of the endogenous Gpx2 gene, wherein each disruption comprises the insertion of a transgene, and wherein the combined disruptions result in a decreased level of GPX-1 and GPX-GI production and decreased number of cells producing GPX-I and GPX-GI in the transgenic mouse as compared to a nontransgenic mouse.

2. A transgenic double knockout mouse as in claim 1 which exhibits one or more physiological symptoms selected from the group consisting of ileitis, colitis, hypothermia, decreased rate of weight gain, perianal ulceration, diarrhea, wasting syndrome, inflammatory bowel disease and cancer of the lower gastro-intestinal tract.

3. A cell isolated from a double knockout mouse as in claim 1 .

4. A cell as in claim 3, selected from the group consisting of a stem cell, an epithelial cell and a myofibroblast.

5. A cell as in claim 4 which is a stem cell.

6. A cell as in claim 4 which is an epithelial cell.

7. A cell as in claim 4 which is a myofibroblast.

8. A transgenic double knockout mouse as in claim 1 which further comprises a mouse which is a germ free mouse.

9. A transgenic double knockout mouse as in claim 1 wherein said knockout mouse is a mouse with a B6 genetic background.

10. A method of selecting an agent for treating a metabolic disorder comprising:
(a) measuring at least one symptom selected from the group consisting of ileitis, colitis, hypothermia, decreased rate of weight gain, perianal ulceration, diarrhea, wasting syndrome, inflammatory bowel disease and cancer of the lower gastro-intestinal tract in a knockout mouse whose genome has been manipulated to comprise a homozygous disruption of both the endogenous Gpx1 gene and Gpx2 genes, wherein the disruption of both the Gpx1 gene and Gpx2 genes results in said knockout mouse exhibiting one or more of said diseases, symptom or symptoms;
(b) administering an agent to said mouse;
(c) measuring one or more of said symptoms in the mouse after administering the agent; and
(d) comparing at least one of said disease, symptom or symptoms in the mouse before and after administering the agent, wherein a decrease in at least one of said diseases, symptom or symptoms after administering the agent indicates the agent is an agent for treating said disease, symptom or symptoms.

11. A method as in claim 10 wherein said knockout mouse is a mouse with a B6 genetic background.

12. A method of selecting an agent that modulates GPX enzyme activity comprising:
(a) administering an agent to a first group of isolated mouse cells and not to a second group of mouse cells, wherein the genomes of both the first and second isolated mouse cell groups have been manipulated to comprise a homozygous disruption of both the endogenous Gpx1 gene and Gpx2 genes, and wherein the disruption of both the Gpx1 gene and Gpx2 genes prevents expression of functional GPX-1 and GPX-GI proteins; and
(b) determining the amount of GPX enzyme activity of the first and second cell groups, wherein a difference in the amount of proliferation of the first cell group as compared to the second cell group indicates that the agent modulates GPX enzyme activity.

13. The method of claim 12 wherein the mouse cells are selected from the group of cell types consisting of stem cells, epithelial cells, intestinal epithelial cells and myofibroblast cells.

14. The method of claim 13 wherein the mouse cells are epithelial cells.

15. The method of claim 14 wherein the epithelial cells are intestinal epithelial cells.

16. The method of claim 13 wherein the cells are stem cells.

17. The method of claim 13 wherein the cells are myofibroblasts.

18. A method of selecting an agent for treating a metabolic disorder comprising:
(a) measuring at least one symptom in a first double knockout mouse having a first genetic background, whose genome is manipulated to comprise a homozygous disruption of both the endogenous Gpx1 and Gpx2 genes, wherein the disruption of both the Gpx1 and Gpx2 genes results in said knockout mouse exhibiting a disease, symptom or symptoms selected from the group consisting of: ileitis, colitis, hypothermia, decreased rate of weight gain, perianal ulceration, diarrhea, wasting syndrome, inflammatory bowel disease and cancer of the lower gastrointestinal tract;
(b) measuring said symptom in a second double knockout mouse having a second genetic background, whose genome is manipulated to comprise a homozygous disruption of both the endogenous Gpx1 and Gpx2 genes, wherein the disruption of both the Gpx1 and Gpx2 genes results in said knockout mouse exhibiting at least one of said disease, symptom or symptoms;
(c) administering an agent to said first and second mouse;
(d) measuring one or more of said symptoms in the first and second mouse after administering the agent; and
(e) comparing at least one of said symptoms in said first and second mouse before and after administering the agent, wherein a decrease in said disease, symptom or symptoms after administering the agent indicates the agent is an agent for treating said disease, symptom or symptoms associated with a metabolic disorder.

19. The method of claim 18 wherein one of said first and second mouse has a B6 genetic background.

20. A transgenic mouse which has a homozygous knockout of the Gpx1 gene and a heterozygous knockout of one allele of the Gpx2 gene.

21. An animal model for the study of the degree of functional redundancy of GPX-1 and GPX-GI in the ileum and colon comprising the mouse of claim 20.

22. A transgenic mouse which has a homozygous knockout of the Gpx2 gene and a heterozygous knockout of one allele of the Gpx1 gene.

23. An animal model for the study of the degree of functional redundancy of GPX-1 and GPX-GI in the ileum and colon comprising the mouse of claim 22.

24. Isolated mammalian cells comprising a diploid genome including chromosomally incorporated transgenes, wherein the transgenes disrupt both alleles of the genomic Gpx1 gene and Gpx2 genes and inhibit expression of said genes.

25. The cells of claim 8, which cells are mouse cells.


SEQUENCE LISTING (5 sequences)

SEQ ID NO: 1 - DNA, 25 bp, Artificial Sequence
  PCR primer for wildtype Gpx1 allele
aaggaggtgcaggcggctgtgagcg

SEQ ID NO: 2 - DNA, 21 bp, Artificial Sequence
  PCR primer for wildtype Gpx1 allele and Gpx1-KO allele
accgttcaccttgcacttctc

SEQ ID NO: 3 - DNA, 26 bp, Artificial Sequence
  PCR primer for Gpx1-KO allele and Gpx2-KO allele
cagtttcatagcctgaagaacgagat

SEQ ID NO: 4 - DNA, 21 bp, Artificial Sequence
  PCR primer for wildtype Gpx2 allele and Gpx2-KO allele
cccacctgtctagaggactta

SEQ ID NO: 5 - DNA, 20 bp, Artificial Sequence
  PCR primer for wildtype Gpx2 allele
tccatgccaacgtagtgatt